(19) United States
(12) Patent Application Publication
(10) Pub. No.: US 2025/0213158 A1
(43) Pub. Date: Jul. 3, 2025

(54) System and Method for Calculating User Satisfaction Indices Based on Multimodal Data

(71) Applicant: Wayvee Inc., New York, NY (US)

(72) Inventors: Aleksei Ovcharov, Limassol (CY); Viacheslav Matiunin, Limassol (CY); Anton Timashev, Limassol (CY); Vitali Mazaleuski, Wysoka (PL); Egor Danilov, Yerevan (AM); Pavel Grachev, Budva (ME); Anna Danilina, Limassol (CY); Dmitrii Mikhailenko, Tbilisi, GA (US); Alexey Nalivaiko, Warsaw (PL); Siarhei Khurs, Warsaw (PL)

(21) Appl. No.: 19/086,978

(22) Filed: Mar. 21, 2025

Related U.S. Application Data
(63) Continuation-in-part of application No. 18/968,599, filed on Dec. 4, 2024, now abandoned.
(60) Provisional application No. 63/606,050, filed on Dec. 4, 2023.

(51) Int. Cl. A61B 5/16; G06V 40/16; G06V 40/20

(52) U.S. Cl. CPC A61B 5/165; G06V 40/174; G06V 40/20

(57) ABSTRACT

A non-invasive, privacy-conscious system and method for calculating User Satisfaction Index (USI) in real-time using multi-sensory data fusion. The system is primarily based on radar sensors, ensuring privacy, while optical and acoustic sensors are optional and can be integrated as needed. It employs machine learning algorithms to analyze physiological (heart rate, respiration, micro-movements), emotional response, and behavioral (gestures, vocal intonation, facial expressions) markers. The User Satisfaction Index prediction and recommendation algorithms estimate users' satisfaction across various industries, including retail, healthcare, corporate environments, smart cities, transportation, and other fields.

### CROSS-REFERENCE TO RELATED APPLICATIONS

This is a continuation in part of application Ser. No. 18/968,599 filed Dec. 4, 2024, which claims the benefit of U.S. patent application Ser. No. 63/606,050 filed Dec. 4, 2023. Each of these applications is incorporated herein by reference for all purposes.

### FIELD

This technology relates to systems and methods for evaluating satisfaction indices (e.g., User Satisfaction Index, USI) using multisensory data obtained from radar, optical, and acoustic sensors. The proposed approach provides non-invasive and efficient evaluation of satisfaction metrics across a wide range of industries, with a strong emphasis on ensuring user data privacy.

### BACKGROUND

Traditional methods for assessing user satisfaction (e.g., surveys and interviews) suffer from subjectivity, limited sample sizes, and delays in obtaining results. Moreover, many known technological solutions (e.g., systems with facial detection and emotion recognition through video) raise concerns about privacy and personal data protection.

The advent of modern sensor technologies offers new opportunities for automated and non-invasive data collection on individuals' physiological and behavioral characteristics. However, existing systems often lack integrated processing modules capable of real-time identification and interpretation of patterns that reflect satisfaction levels.

### OVERVIEW OF SOME EXISTING SOLUTIONS

Conventional methods for evaluating user satisfaction primarily rely on subjective surveys, interviews, and questionnaires, which face several limitations:
Inability to provide real-time data;
Risk of subjective biases and inaccurate responses;
Challenges in scaling such surveys;
Delays in identifying issues related to declining satisfaction.

Modern systems leveraging biometric and behavioral signals (e.g., video, voice, gestures, heart rate, etc.) are gaining traction. Specifically:
U.S. Pat. No. 10,256,567 B2 describes real-time speech analysis for emotion recognition, closely related to acoustic monitoring;
US 2017/0112169 A1 provides examples of a multisensory approach (optical and acoustic sensors) for detecting emotional states;
U.S. Pat. Nos. 8,619,201 B2, 8,920,332 B2, US 2016/0338421 A1, and EP 3294872 A1 extensively detail non-invasive methods for measuring physiological signals (heart rate, respiration) using radar;
U.S. Pat. No. 9,363,498 B2 describes a system for collecting human physiological data to enhance safety or comfort, without focusing on multidimensional satisfaction evaluation.

In addition to patents, scientific publications (e.g., A microwave radio for Doppler radar sensing of vital signs, DOI: 10.1109/MWSYM.2001.966866) demonstrate the use of radar for contactless measurement of breathing and heart rate. Research on emotion recognition through audio and video data (e.g., Soleymani M. et al., 2012; Picard R., 1997; Cowie R. et al., 2001) confirms the effectiveness of multimodal analysis.

However, these solutions are either focused on narrow tasks (e.g., measuring breathing) or limited to audio or video analysis, which may raise privacy concerns. Few existing developments offer an integrated framework where radar serves as the primary “private” data source, with optical and acoustic sensors used optionally. Furthermore, they do not prioritize calculating User Satisfaction Index (USI) across various applied scenarios.

### BRIEF DESCRIPTION OF THE DRAWINGS

FIG. 1 is a General System Diagram.

FIG. 2 is a sensor fusion and synchronization block diagram.

FIG. 3 is a tracking block diagram.

FIG. 4 shows target behavior and activity classification.

FIG. 5 shows an example practical application.

FIG. 6 is a block diagram of an example implementation.

FIG. 7 shows a layered architecture.

FIG. 8 is a block diagram of an example sensor.

### TECHNICAL PROBLEM AND ITS SOLUTION

A problem addressed by the example technology herein is the development of a comprehensive, non-invasive, privacy-conscious system capable of real-time USI calculation through:
Prioritized use of radar sensors to ensure privacy;
Optional and configurable integration of optical and acoustic sensors;
Intelligent data fusion algorithms and machine learning to analyze physical (heart rate, respiration, micro-movements), emotional response and behavioral (gestures, vocal intonation, facial expressions) markers;
Generating predictions and recommendations for timely improvement of satisfaction levels.

### DETAILED DESCRIPTION OF EXAMPLE NON-LIMITING EMBODIMENTS

User Satisfaction Index is a quantitative metric used to evaluate the overall satisfaction of individuals interacting with a service, product, or environment. Our system derives USI dynamically through real-time physiological, behavioral, and contextual data analysis. The index represents an objective assessment of a user's emotional and psychological state while interacting with a given service, company, or environment.

In conventional methods, USI is computed from post-experience feedback, often suffering from biases and limited sample sizes. Our example embodiment approach overcomes these limitations by leveraging multisensory data fusion to infer satisfaction in real-time without directly asking for post-experience feedback.

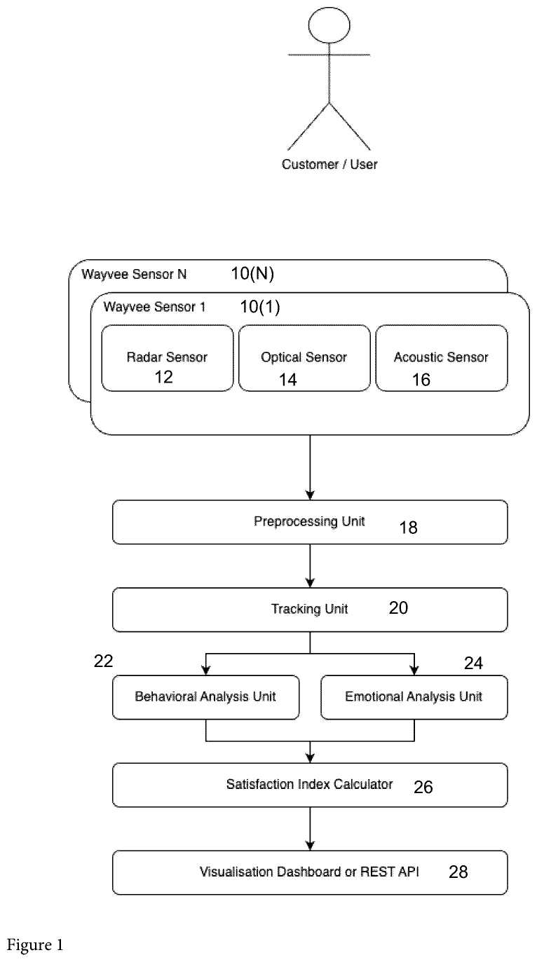

An example embodiment for real-time calculation of satisfaction indices (USI) operates as depicted in FIG. 1 and consists of the following components:
Multisensory Input Modules: These modules, labeled as Wayvee Sensors 10(1), . . . , 10(N), may include radar sensor(s) 12, optical sensor(s) 14, and acoustic sensor(s) 16. They are designed to provide data with flexible privacy modes. Radar sensors are prioritized to ensure privacy, while optical and acoustic sensors can be activated based on user consent for deeper analytics.
Preprocessing Unit 18: This unit processes the raw input data by applying noise filtering, normalization, and synchronization techniques. It prepares the data for further analysis.
Tracking Unit 20: The tracking unit processes data from the sensors to continuously monitor movements, positions, and other key metrics, forming the basis for behavioral and emotional analysis.
Behavioral and Emotional Analysis Unit(s):
   The Behavioral Analysis Unit 22 focuses on extracting features like body movements, gestures, posture, and micro-movements.
   The Emotional Analysis Unit 24 analyzes vocal patterns, facial expressions, and emotional indicators derived from acoustic, optical, and physiological data.
Satisfaction Index Calculator 26: Utilizing machine learning algorithms, this module computes the USI. It considers biometric, behavioral, and contextual factors such as crowd density, noise levels, and lighting.
Visualization Dashboard or REST API 28: Provides real-time monitoring of USI and enables access to recommendations for improving satisfaction levels. The dashboard also supports integration with external software via a REST API.

The example system emphasizes privacy by prioritizing radar sensors 12 to collect key physiological metrics and includes options to disable or mask optical and acoustic data. The system directly uses analytical results for USI computation, capturing the user's current state while enabling prediction and notification mechanisms.
Privacy-Centric Design: Radar sensors are the primary data source, reducing the need for sensitive visual or acoustic data. Privacy-enhancing mechanisms such as anonymization and masking are available.
Real-Time Capabilities: The system offers instant computation and visualization of satisfaction metrics, supporting predictive analytics and actionable recommendations.
Flexibility: Modular architecture allows the system to adapt to various environments, including retail, healthcare, and smart city applications.

### 1. Multisensory Data Collection Modules

An example embodiment employs three primary types of sensors—radar, optical, and acoustic—to gather real-time data about human physiological and behavioral parameters. Each sensor type is outlined below.

### 1.1 Radar Sensors 12

### Primary Function:

Radar serves as the main source for measuring body motion, breathing rate, heart rate (HR), heart rate variability (HRV), and micro-movements.

### Technology:

The system uses Frequency-Modulated Continuous Wave (FMCW) radar with an antenna array employing MIMO (Multiple Input Multiple Output) technology, which incorporates multiple transmitting and receiving antennas operating in the 1-100 GHz range. Our advanced algorithms also process data from an array of virtual antennas, enabling detection of subtle motions like limb gestures, skin movement during heartbeat, and chest displacement from breathing.

### Privacy Clarification:

The radar signals do not encode facial images or other visually identifying features. While HR, HRV, and breathing rate can be considered sensitive, they are not tied to a person's identity without additional cross-referencing. Thus, radar data is treated as an anonymized physiological signal, reducing privacy concerns compared to full video capture.

### 1.2 Optical Sensors (Optional) 14

### Types:

RGB cameras, infrared (IR) sensors, and depth (3D) sensors.

### Purpose:

Optical sensors can complement radar data by recognizing gestures, facial expressions, posture, and spatial interactions, increasing context and accuracy when privacy settings allow.

### Optional Privacy Modes:

1. Pixelation of Faces: Automatically blurs faces in live or recorded video.
2. Anonymization: Dims or obscures specific regions within the frame to avoid capturing identifiable details.
3. Automatic Stream Deactivation: Disables video capture when a defined privacy threshold is exceeded or user preference indicates.

### 1.3 Acoustic Sensors (Optional) 16

### Frequency Range:

20 Hz-20 kHz, covering the typical range of human speech and many ambient environmental sounds.

### Functions:

1. Vocal Signal Analysis: Detects stress or emotional changes through voice pitch, timbre, rhythm, and pause distribution.
2. Acoustic Anomaly Detection: Identifies sudden changes in the acoustic environment, such as shouting or sharp noises, which could signify elevated stress or dissatisfaction.
3. Real-Time Transcription: Converts speech into text using Automatic Speech Recognition (ASR) for structured verbal analysis.

### Directional Capture:

Beamforming pinpoints individual voices or sound sources, thus reducing overall background noise and enabling more targeted analysis of vocal patterns.

### 2. Data Preprocessing Unit 18

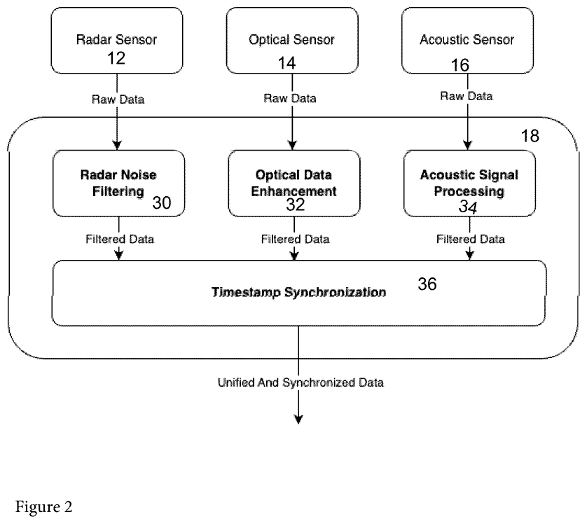

As shown in FIG. 2, raw radar, optical, and acoustic data are ingested by the Data Preprocessing Unit, which ensures consistent and noise-reduced inputs for subsequent analysis. The data preprocessing unit may comprise one or more processors such as CPUs and/or GPUs executing instructions stored in memory, and/or one or more application specific integrated circuits (ASICs), or a combination of these.
1. Radar Noise Filtering 30:
   Adaptive low-pass and high-pass filters remove high-frequency noise and low-frequencies corresponding to short range information.
   An interference handling step identifies and eliminates interference between multiple radars operating within the same environment.
2. Optical Data Enhancement 32:
   Deblurring and low-light enhancement improve visibility, while optional masking or blurring of sensitive regions (e.g., faces) can be applied for privacy.
3. Acoustic Signal Processing 34:
   Spectral subtraction and phase correction reduce background noise.
   Beamforming isolates specific voices for emotional stress detection.
4. Timestamp Synchronization 36:
   When multiple sensors are active, data streams are time-aligned, ensuring radar, optical, and acoustic events are correlated accurately.

### 3. Tracking Unit 20

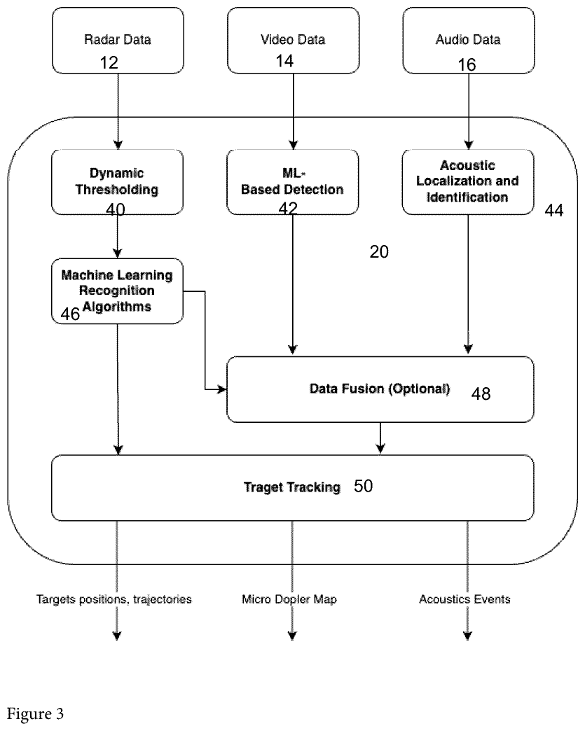

As shown in FIG. 3, an example embodiment includes a Target Recognition and Tracking Unit 20 capable of detecting, identifying (in a non-personally identifying manner), and monitoring human targets using radar, optical (optional), and acoustic (optional) sensors 12, 14, 16.

### 3.1 Radar Target Recognition

To extract spatial information about the environment the Range-Azimuth, Range-Azimuth-Elevation and Angle of Arrival maps are calculated from Raw Intermediate Frequency Data and analyzed together with Doppler FFT Output and Range-Doppler Map.

### Dynamic Thresholding: 40

Thresholds adapt to variations in the environment (e.g., changes in object density or background motion), minimizing false positives.

### Machine Learning Recognition Algorithms: 46

Classification or clustering methods recognize patterns associated with individual human targets. Although the term “recognize” is used, the system does not identify personal details; rather, it distinguishes one moving target from another. Such classification or clustering methods may be performed by one or more machine learning components such as one or more trained neural networks.

### 3.2 Optical Target Recognition (Optional)

### ML-Based Detection 42:

Visual algorithms detect persons within the camera's field of view, optionally applying body or skeleton tracking in 2D or 3D. Such visual algorithms may be performed by one or more machine learning components such as one or more trained neural networks.
   Data Fusion with Radar 48:
When both radar and optical sensors are employed, fused data can enhance detection reliability under complex or crowded conditions.

### 3.3 Acoustic Target Recognition (Optional)

### Acoustic Localization and Identification 44:

With acoustic sensors, the system can approximate where a sound (e.g., a voice, a loud noise) originates.
   Beamforming pinpoints specific voices or sound events among multiple audio sources, aiding in localizing the source of speech or unusual noises.
   Voice Signature (non-identifiable): Distinguishes one speaker's characteristics from another's, without linking them to personal identity.

### Data Fusion (Radar+Optical+Acoustic) 48:

Combining three modalities facilitates more robust recognition, especially when multiple individuals are present. For instance, the acoustic sensor can indicate who is speaking or reacting to an event, while radar and optical streams capture movement patterns.

### 3.4 Target Tracking 50

### Continuous Monitoring:

Once a target is recognized, its position, trajectory, and activity are tracked over time.
   Integration with Acoustic Cues (Optional):
Acoustic events like sudden loud sounds, shouting, or distinct footsteps can be associated with a particular radar/optical track.
Any anomaly (e.g., a sharp cry without accompanying radar-detected movement) can trigger further system analysis.

### Micro-Doppler Map:

From the radar data, a Micro-Doppler map is generated and analyzed together with other Radar-based information to reveal fine-grained body movements (e.g., limb oscillations). This feature is particularly valuable for distinguishing various types of motion (walking, running, etc.) or activities (e.g., falling).

### 4. Target Behavior and Activity Classification 52

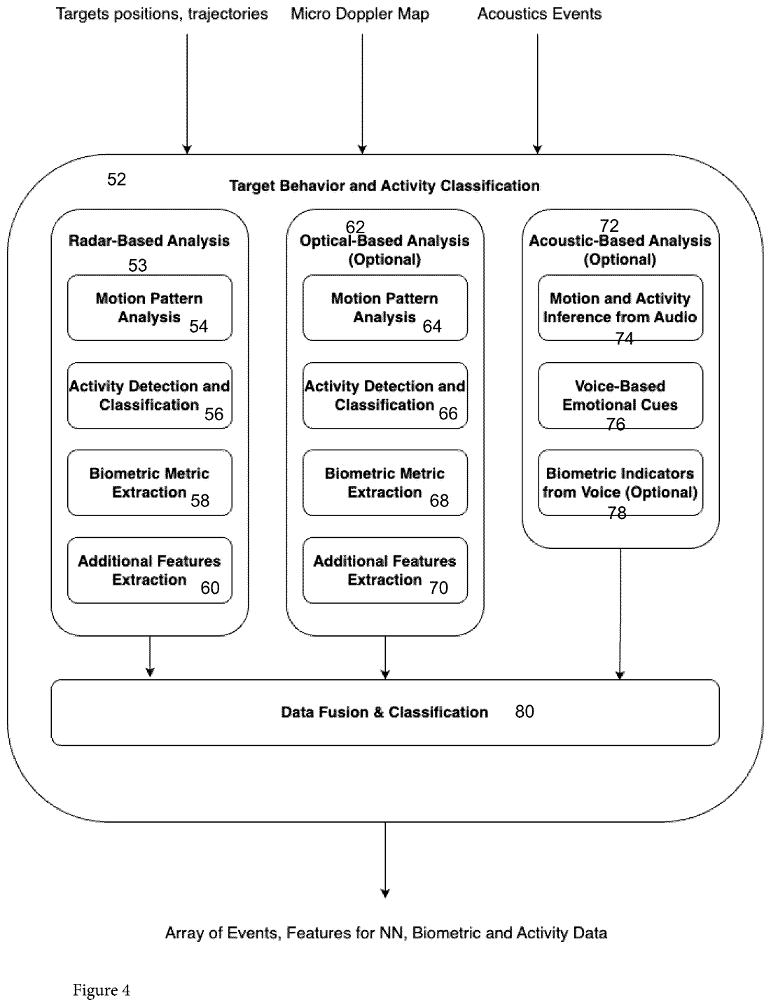

As shown in FIG. 4, after detection and tracking, the system classifies the target's activities and extracts various biometric and behavioral metrics. This process can be accomplished using radar, and/or optical, and/or acoustic channels, depending on system configuration.

### 4.1 Radar-Based Analysis 53

1. Motion Pattern Analysis 54:
   Electromagnetic signal variations due to doppler shift reveal walking speed, direction, acceleration, and subtle micro-movements.
2. Activity Detection and Classification 56:
   Machine learning models categorize common actions (waving, running, falling, object interaction) by analyzing Doppler-based signatures of moving body parts. Such machine learning models may be implemented as one or more neural networks executing on one more processors.
3. Biometric Parameters/Metrics Extraction 58:
   Heart Rate (HR), Heart Rate Variability (HRV), and breathing patterns are derived from radar signals with high temporal resolution.
   While these metrics are sensitive to physiological changes, they do not reveal an individual's identity.
4. Additional Features Exaction 60:
   Advanced algorithms combine kinematic parameters (e.g., stride length, motion smoothness) and biometric signals (HR, breathing amplitude) to form a holistic user profile.

### 4.2 Optical-Based Analysis (Optional) 62

1. Motion Pattern Analysis 64:
   Optical pose estimation locates body parts in 2D or 3D space, enabling calculation of speed, direction, and changes in posture.
2. Activity Detection and Classification 66:
   Skeleton-tracking or ML-based recognition identifies actions like waving, running, or interacting with objects in detail.
3. Biometric Metric Extraction 68:
   If IR or near-infrared (NIR) data is available, approximate vital signs (e.g., HR via photoplethysmography) can be extracted.
4. Additional Features Extraction 70:
   Facial expressions, micro-expressions, or gestures can supplement radar signals, provided user privacy settings permit and radar resolution.

### 4.3 Acoustic-Based Analysis (Optional) 72

1. Motion and Activity Inference from Audio 74:
   In quieter settings, certain activities produce distinct sound patterns—footsteps, falling objects, door creaks, etc.
   ML algorithms classify these audio signals, identifying potential states like “walking,” “running,” “shouting,” or “applause.”
2. Voice-Based Emotional Cues 76:
   Changes in pitch, loudness, speech rate, and pause frequency can indicate stress, excitement, irritation, or calm.
   Beamforming enables isolating a specific speaker to evaluate emotional state without requiring explicit identity recognition.
3. Biometric Indicators from Voice (Optional) 78:
   Breathing rhythm may be partially inferred if the microphone captures inhalation/exhalation patterns.
   While less precise than radar or optical methods, it can still enhance confidence in overall activity or stress classification.

### 4.4 Data Fusion and Consistency Checks 80:

If acoustic events (e.g., a loud exclamation) coincide with radar/optical-detected gestures (e.g., abrupt arm movement), the confidence in labeling an “agitated” or “excited” state increases.
Conversely, mismatches (noise without physical movement) could flag anomalies needing additional evaluation.

The above operations may be performed by a computing platform or computing instance comprising for example at least one processor such as a CPU or GPU, that executes instructions stored in non-transitory memory.

### 5. Satisfaction Index Calculation

A core objective of certain non-limiting embodiments is to compute real-time satisfaction metrics—particularly the User Satisfaction Index (USI)—based on physiological, behavioral, and, optionally, acoustic features.

### 5.1 User Emotional Response Estimation 24

Radar-Only: The machine learning model uses time-series data (HR, HRV, breathing patterns, micro-motion and behavior and activity classification data) to infer emotional states. The ML algorithms are trained using empirical data f, where auxiliary sensors (e.g., GSR, HR, Breathing belt, eye-tracker) and user surveys provide ground truth for emotional labeling. The ML algorithms may be implemented by one or more neural networks executing on one or more processors.
Multimodal (Radar+Optical+Acoustic): When optical and acoustic data are also available, the system integrates facial expressions, poses, vocal tone, and speech patterns to achieve a more nuanced understanding of emotional and satisfaction levels.
Audio-Based Stress and Emotion Indicators: If the acoustic sensor is active, features such as pitch, volume, speech rate, and specific word usage (if speech recognition is employed) are fed into the model. The system may operate in a speaker-anonymized manner, ensuring no personal voice identification is performed.

### 5.2 USI Calculation Algorithms

### Aggregate Metrics:

The satisfaction index derives from a weighted combination of physiological (HRV, breathing), behavioral (gestures, micro-expressions), acoustic (voice stress, loudness), and contextual (noise level, crowd density) parameters.
The algorithm can detect signs of discomfort, frustration, or high engagement.

### Feedback Integration:

Subjective user ratings (e.g., quick surveys) provide ground-truth updates to retrain or calibrate the model, ensuring continuous refinement.

Linguistic analysis (optional) can track positive or negative words in speech to further validate dissatisfaction or satisfaction cues.

The final USI score is normalized within a 0-100 range, where:
0-40: Dissatisfaction detected (e.g., frustration, discomfort)
   41-60: Neutral state (e.g., neither strong satisfaction nor dissatisfaction)
   61-100: Satisfaction detected (e.g., positive engagement, relaxation)
Example Use Cases:
   Retail: USI below 40 may indicate long checkout delays, prompting additional staff allocation.
   Healthcare: USI tracking in waiting rooms helps detect patient stress, allowing proactive support.
   Workplaces: Persistent low USI among employees signals burnout risk, enabling HR intervention.

### 5.3 Data Collection and USI Labeling Process

The reliability of USI estimation in our system is directly linked to how the dataset is created and labeled. Our approach follows these key steps:

### 5.3.1 Multisensory Data Acquisition

USI is determined by aggregating various physiological, behavioral, and contextual parameters obtained from the system's sensors and postprocessing of the raw data.

### 5.3.2 Ground Truth Labeling for USI Model Training

Since satisfaction is inherently subjective, creating an accurate dataset for training requires well-defined ground truth labels. The labeling process follows a multi-step approach:
1. Empirical data from Controlled Experiments with Verified Labels
   Participants interact with predefined service scenarios (e.g., retail shelf, customer service counters, self-checkout kiosks, hospital waiting rooms).
   Participants' self-reported satisfaction levels are collected immediately after each interaction using a standardized scale (e.g., a Likert scale from 1 to 10).
   To further validate emotional states, auxiliary sensors—such as Galvanic Skin Response (GSR), heart rate monitors, breathing belts, and eye-trackers—are used in parallel. These additional physiological and behavioral signals help confirm the participants' self-reported satisfaction levels.
   By combining self-reports and data from these auxiliary sensors, the system can more accurately correlate radar (and, optionally, optical/acoustic) sensor readings with reliable satisfaction scores.
2. Correlation with Industry-Specific Satisfaction Metrics
   In business environments, external benchmarks such as Net Promoter Score (NPS) or Customer Effort Score (CES) are incorporated to enhance USI model training.
   Behavioral trends of highly satisfied and dissatisfied individuals are analyzed in relation to existing service quality metrics.
3. Real-World Unsupervised Data Collection
   The system continuously collects and processes sensor data from deployed environments.
   Machine learning models assign initial USI scores based on previously labeled datasets.
   An optional feedback loop allows users (customers, employees) to confirm or adjust inferred satisfaction levels, refining the model over time.

### 6. Visualization Dashboard

The system provides real-time feedback to users and stakeholders through a dashboard interface.

### 6.1 Real-Time Metrics

USI Graphs: Display instantaneous and historical satisfaction levels for quick trend analysis.
Heatmaps: Visualize spatial distribution of stress or satisfaction, highlighting areas needing attention.
Integrated Reports: Allow comparisons across different time frames, locations, or user segments, enabling robust analytics.

### 6.2 Notifications and Recommendations

Automatic Alerts: A sudden drop in USI triggers notifications to management or designated personnel, prompting rapid intervention (e.g., reducing noise, adding staff).
Prescriptive Analytics: Suggests actions—adjusting temperature, optimizing layout, altering background music volume—to improve overall satisfaction.
Multiplatform Access: The dashboard is accessible via mobile, web, or enterprise platforms, facilitating decision-making at multiple organizational levels.

### EXAMPLE OF DISTRIBUTED SENSORS SYSTEM DEPLOYMENT

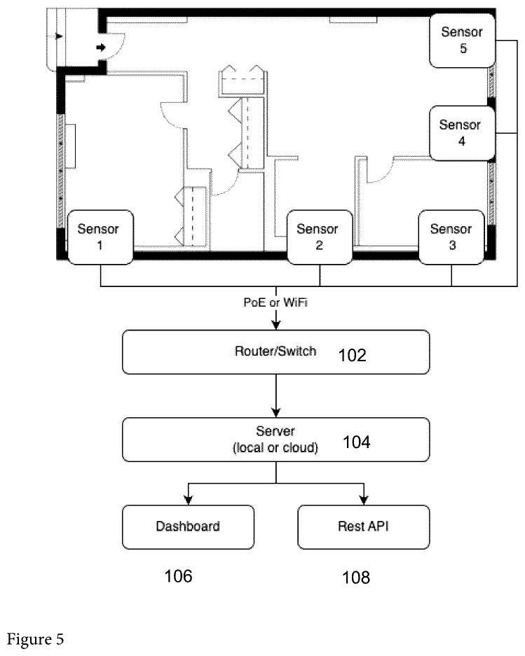

FIG. 5 illustrates an example configuration of the proposed distributed sensors system deployed across a defined area. The system is designed to monitor environmental conditions, behavior patterns, and satisfaction levels in real-time using strategically placed sensors and centralized processing.

### System Components and Workflow:

### Distributed Sensors 200:

Multiple sensors (e.g., Sensor 1, Sensor 2, Sensor 3, Sensor 4, and Sensor 5) are strategically placed throughout the area to cover key zones such as entry points, high-traffic regions, and specific points of interest.
Each sensor integrates radar, optical, and acoustic modules for collecting multisensory data, including movement, biometric signals, and acoustic/environmental factors.

### Connectivity:

The sensors communicate with the system infrastructure via Power over Ethernet (PoE) or WiFi, offering deployment flexibility and reducing the need for extensive cabling.

### Router/Switch 102:

Data streams from all sensors are aggregated through a router or switch, ensuring seamless data transmission to the processing unit.

### Server 104:

Aggregated data is transmitted to a central server, which can be deployed locally within the area of deployment or on a cloud platform based on system requirements.
The server processes raw sensor data through advanced modules such as preprocessing, tracking, and analysis to compute relevant metrics (e.g., satisfaction indices, behavioral insights).

### Visualization and Integration:

Processed data is accessible through two main interfaces:
   1. Dashboard 106: A real-time monitoring tool for visualizing key metrics such as behavioral trends, satisfaction indices, and environmental parameters.
   2. REST API 108: Allows integration with third-party systems for further analytics, reporting, and automated actions.

### EXAMPLE

### 1. System Overview

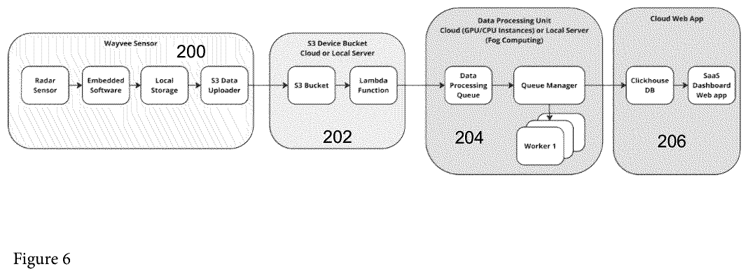

An example embodiment of a system shown in FIG. 6 is designed to collect and analyze physiological and behavioral data in real time in one or more stores (or any other locations). Sensors (radar modules, optionally with optical sensors) are placed in the facility, connect to the local network (Wi-Fi or Ethernet), and then transmit the collected information to the cloud or a local server for further processing.

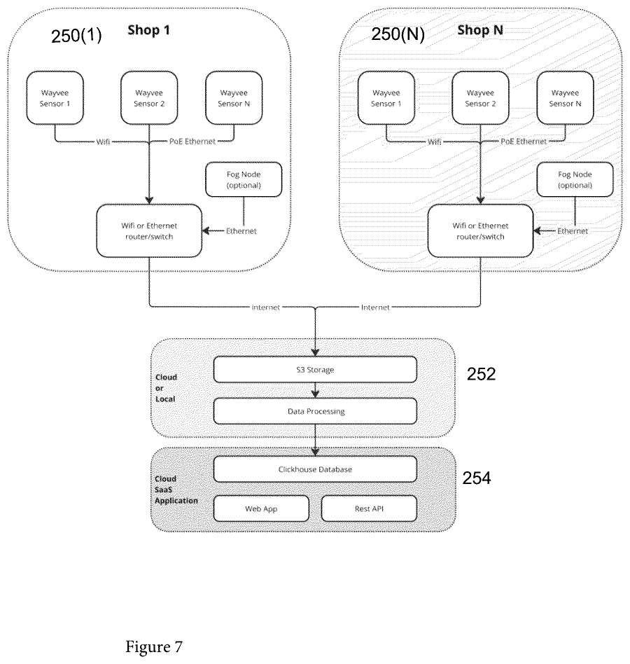

We can conceptually divide the system into the following layers as shown in FIG. 7:

Store-Level (Local) Environment 250 (1), 250 (N):

A group of Wayvee sensors in a single physical space.

A Wi-Fi/Ethernet network, and an optional Fog Node for local pre-processing.

Cloud or Local Infrastructure (S3 Bucket, Queues, Processing):

A storage location (S3 or an S3-compatible solution) for raw data.

A trigger mechanism (e.g., Lambda Function) to place new data items into a processing queue.

A pool of workers (in the Cloud or Fog) that perform ML/analytics tasks.

Database and Web Application (ClickHouse, SaaS Dashboard):

A high-performance analytical database.

A web interface or REST API for displaying results and integrating with third-party systems.

### 2. Internal Structure of the Wayvee Sensor

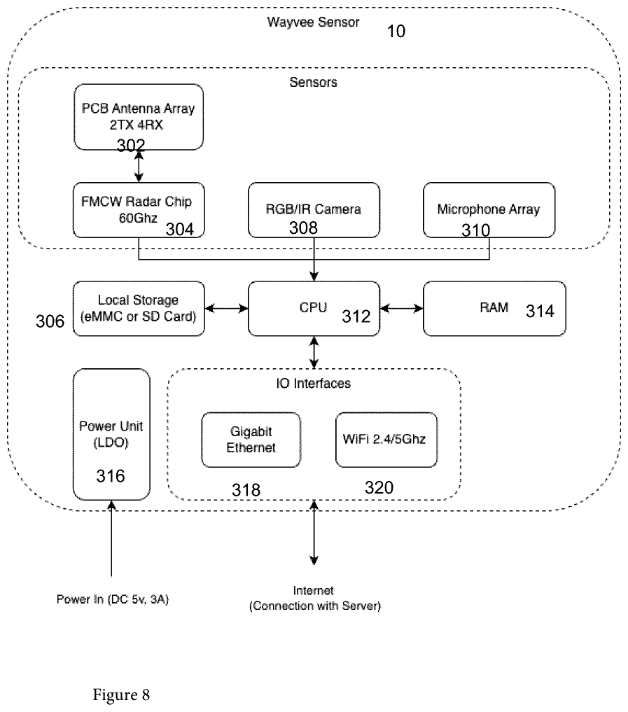

The FIG. 8 diagram shows a modular sensor design that merges multiple sensing technologies (radar, camera (optionally), microphone array (optionally)) with an embedded CPU and storage. Below are the key components:
1. FMCW Radar Chip (60 GHz) 304: Core Radar Module that transmits and receives frequency-modulated continuous-wave signals.Operates in the 60 GHz band enabling fine-grained detection of movements, breathing, or micro-gestures.
2. PCB Antenna Array (2TX/4RX) 302: Printed antenna system specifically tuned for the 60 GHz radar chip. Two transmitting elements (2TX) and four receiving elements (4RX) enhance spatial resolution and sensitivity to small physiological changes.
3. RGB/IR Camera (Optional): 308 Visual sensor that can capture standard RGB footage or infrared imagery. Often used to add contextual or safety information (e.g., facial expressions, occupancy monitoring). If deployed, it integrates with the CPU for synchronized data capture alongside radar measurements.
4. Microphone Array (Optional) 310: A set of microphones enabling audio-based detection, such as voice activity or ambient noise analysis. Can also perform beamforming or direction-finding if needed, depending on the application software.
5. CPU and RAM

The embedded processor 312 (e.g., an ARM-based system) handles control, data pre-processing, and communication tasks.

RAM (system memory) 314 is used to run embedded software, including radar drivers, camera pipelines, and local analytics modules.
6. Local Storage (eMMC or SD Card) 306: Onboard data storage for buffering, caching, or partial analytics results. Protects against data loss if the network is unavailable (e.g., storing sensor data until connectivity resumes).
7. IO Interfaces

Gigabit Ethernet 318: For reliable wired communication and potential Power over Ethernet (PoE).

Wi-Fi (2.4/5 GHZ) 320: Wireless connectivity for installations where running cables is impractical.

Both options may be present, allowing flexible deployment in diverse environments.
8. Power Unit (LDO) 316

Low-Dropout Regulator (LDO) that provides stable voltage supply to all internal components.

Receives DC input at 5 V, 3 A, ensuring enough power for radar, CPU, camera, and other modules.
3. End-to-End Data Flow (see FIG. 6)

Radar Collection 200: The Radar Sensor reads high-frequency signals; a microcontroller or embedded software digitizes and applies basic processing (noise filtering, spectral transformation).

S3 device bucket 202 cloud or local server; includes an S3 bucket and a lamda function

Local Processing 204: Software on the Wayvee Sensor may further compress the data, add metadata (timestamps, serial IDs), and perform preliminary analytics (breathing cycle estimation, movement detection).

Temporary Storage: The resulting fragments (files or data blocks) are stored in local memory on the wayvee sensor

S3 Upload 206: When network connectivity is available, the sensor initiates data transfer to cloud/local S3 storage.

Queue and Further Processing: Once the files appear in the Bucket, a serverless function creates tasks in a queue. Workers then process these tasks—e.g., ML, anomaly detection, or feature extraction.

ClickHouse and Analytics: Final metrics are logged in ClickHouse, from which they can be queried in real time by the SaaS Dashboard or REST API.

### Industrial Applicability

1. Retail: Evaluation of visitor satisfaction in retail spaces, considering crowd distribution, service quality, marketing activities, A/B Tests, Price Elasticity Research.
2. Healthcare: Monitoring the psychophysiological states of patients and staff (e.g., stress, fatigue, satisfaction with conditions).
3. Transportation: Enhancing service quality in airports, train stations, and public transport by early detection of passenger dissatisfaction.
4. Workspaces: Monitoring employee well-being, stress levels, and motivation to facilitate timely HR interventions.
5. Education: Analyzing engagement and emotional states of students in classrooms and online learning environments.
6. Smart Cities: Assessing citizen satisfaction with public spaces and services (e.g., parks, public events).
7. Security: Detecting behavioral anomalies in high-security zones (e.g., airports, industrial facilities).

And other institutions across diverse fields including retailers, banking, social services, healthcare, government, education, transportation and other businesses and nonprofit organizations that are interested in insights from their target audiences, such as customers, employees, citizens, and students, on a broad range of issues.

### Conclusion

The proposed system and method for calculating User Satisfaction Index (USI) based on multisensory data (radar, optical, and acoustic) enable non-invasive and confidential real-time measurement of objective emotional state metrics. This solution is relevant for numerous industries—from retail and healthcare to smart cities and educational institutions—and unlocks new opportunities for analyzing and improving user satisfaction. Example non-limiting embodiments provide enhanced privacy through the prioritized use of radar sensors and additional anonymization mechanisms for optical and acoustic data. The extended system also provides predictive analytics and recommendation functionalities, proactively improving USI scores and overall service quality and operational processes.

### EXAMPLE REFERENCES

### 1. Patents and Patent Publications

1. U.S. Pat. No. 8,619,201 B2: Sensing device using a microwave radar for measuring vital signs
2. Describes the use of microwave radar for non-invasive monitoring of physiological indicators (e.g., heart rate, respiration). Provides a foundation for radar-based human state tracking.
3. U.S. Pat. No. 9,363,498 B2: System and method for real-time physiological monitoring of an occupant in a vehicle
4. Proposes a sensor-based system for continuous monitoring of passenger states, including heart rate and respiration, demonstrating a multisensory approach in confined spaces.
5. U.S. Pat. No. 8,920,332 B2: Noncontact heart rate measuring system and method
6. Describes a contactless heart rate measurement method, aligning with radar-based and non-invasive principles.
7. U.S. Pat. No. 10,256,567 B2: Emotion detection using speech analysis in real time
8. Claims a system for speech analysis to determine emotional states (e.g., stress, happiness), relevant to the acoustic analysis component of the patent.
9. US 2017/0112169 A1: Method and system for emotion recognition based on multiple sensors
10. Describes a multisensory approach (including optical and acoustic sensors) for emotion recognition, exemplifying integrated data analysis.
11. US 2016/0338421 A1: System for continuous non-contact measuring of vital signs in real time using Doppler radar
12. Demonstrates the application of Doppler radar for vital sign measurement, a key component of the discussed system.
13. EP 3294872 A1: Methods and devices for non-contact vital signs monitoring using radar
14. Focuses on methods for monitoring respiration and heart rate non-invasively, serving as a strong European example in radar technologies.

### 2. Scientific Publications and Articles

1. Adib F. et al., “Vital-Radio: Tracking Vital Signs Using Wireless Signals,” Proceedings of the ACM Conference on Mobile Computing and Networking (MobiCom), 2015.
2. Li C. et al., “Microwave Doppler radar for accurate respiration characterization and personalized healthcare,” IEEE Journal of Electromagnetics, RF and Microwaves in Medicine and Biology, 2017.
3. Soleymani M., Pantic M., Pun T., Picard R., “A Survey of Affect Recognition Methods: Audio, Visual, and Spontaneous Expressions,” IEEE Transactions on Pattern Analysis and Machine Intelligence, 2012.
4. Picard R., “Affective Computing,” MIT Press, 1997.
5. Amin M. G. (Ed.), “Radar for Indoor Monitoring: Detection, Classification, and Assessment,” CRC Press, 2017.
6. Cowie R., Douglas-Cowie E., et al., “Emotion recognition in human-computer interaction,” IEEE Signal Processing Magazine, 2001.

### 3. Commercial and Prototype Solutions

1. Google Nest Hub (2nd generation)/Project Soli: Demonstrates the use of radar technology for contactless sleep monitoring (e.g., breathing analysis, micro-movements).
2. Affectiva (A Subsidiary of Smart Eye): Develops software solutions for emotion recognition using video (optical input) and audio, showcasing multisensory emotional analysis practices.
3. Intel RealSense: A 3D camera technology with SDK for gesture, facial, and volumetric object recognition, exemplifying optical solutions in user and industrial scenarios.
4. Emotient (acquired by Apple), Affectiva, Noldus FaceReader: Commercial and scientific solutions for facial expression and emotion analysis, confirming the market demand for multimodal emotional state recognition systems.

1. A system for real-time calculation of a user satisfaction index (USI), the system comprising:
multisensory input sensors including:
a non-contact radar sensor configured to measure physiological and behavioral parameters comprising at least body motion, breathing rate, heart rate, heart rate variability, and micro-movements; and
at least one processor connected to the radar sensor and configured to perform operations comprising:
filter noise from, normalize, and synchronize data derived from the radar sensor;
analyze user data using a tracking module to continuously monitor user movements, positions, and other metrics that form the basis for behavioral and emotional analysis;
extract biometric and behavioral features from the filtered, normalized and synchronized data using a behavioral and emotional analysis unit; and
compute, via at least one machine learning algorithm, a USI indicative of a user's real-time satisfaction state based on biometric and behavioral features;
an interface circuit connected to the at least one processor and configured to perform operations comprising:
generate, based on the computed USI, signals for a visualization dashboard and/or a REST API to display the USI; and
enable integration of the computed USI with external systems or software for further analysis or for issuing recommendations to improve the user's satisfaction state.

2. The system of claim 1, wherein the non-contact radar sensor is a primary data source to ensure privacy, with optical and acoustic sensors activated upon user consent for deeper analytics.

3. The system of claim 1, wherein the non-contact radar sensor measures physiological parameters, including heart rate (HR) and respiratory rate, using phase and amplitude modulation of signals.

4. The system of claim 1, wherein the non-contact radar sensor is capable of detecting gestures, kinematic parameters of the human body, position, velocity in space, and spatial interactions with environment.

5. The system of claim 1, further including optical sensors configured to detect gestures, facial expressions, and spatial interactions, with pixelation and anonymization for enhanced privacy.

6. The system of claim 1, further including acoustic sensors configured to analyze vocal and sound patterns to determine emotional states based on acoustic features and background noise.

7. The system of claim 1, further comprising a hardware-software complex for predictive analytics, capable of forecasting satisfaction level changes and generating recommendations for improving USI.

8. The system of claim 1, wherein the system is designed for scalable deployment, allowing the installation of multiple sensors across large spaces such as retail stores, office environments, or public areas.

9. The system of claim 8, wherein each sensor operates independently but synchronizes data through a central server or cloud infrastructure for integrated analysis.

10. The system of claim 8, further comprising a setup and configuration module that automates sensor placement calibration, ensuring optimal data capture and alignment within the monitored environment.

11. The system of claim 8, wherein sensor scalability supports dynamic network adjustments, allowing addition or removal of sensors without disrupting ongoing operations.

12. The system of claim 1, wherein data processing is supported across multiple architectures, including:
Cloud Computing comprising Centralized processing on cloud servers for scalability and large-scale data integration,
Fog Computing comprising Local server-based processing for reduced latency and efficient resource use.
Edge Computing comprising On-device processing for real-time analytics and enhanced privacy.

13. The system of claim 12, wherein a processing architecture is dynamically selected based on network conditions, computational requirements, or user preferences.

14. A method for calculating satisfaction indices, comprising:
Collecting multisensory data including collecting radar data from radar sensors;
Preprocessing the collected radar data to filter noise, standardized the data, and synchronize the data
With at least one processor, Extracting emotional and behavioral features from the filtered, standardized and synchronized data using analysis algorithms;
Based at least in part on the extracted emotional and behavioral features, Calculating a satisfaction index (USI) via a machine learning algorithm; and
Displaying the calculated satisfaction index on a visualization dashboard, generating recommendations, and/or providing data access through a REST API.

15. The method of claim 14, wherein the method accounts for individual user parameters and environmental context factors.

16. The method of claim 14, further comprising adaptively learning including retraining a machine learning model on new data to improve satisfaction level prediction accuracy.

17. The method of claim 14, wherein the visualization dashboard and REST API interface include automatic notifications and recommendations for adjusting environmental factors, preferably temperature, lighting, and noise, that impact satisfaction.

18. The method of claim 14, further comprising placing and calibrating sensors, and networking the sensors based on predefined spatial analysis and environmental requirements.

19. The method of claim 14, further including achieving scalability through modular configurations, allowing real-time adjustments to the number and placement of sensors depending on the monitored area's size and density.

20. The method of claim 14, wherein processing the radar data comprises executing on:
Cloud servers for aggregated large-scale analysis;
Local fog servers to ensure low-latency and near-site computation; and
Edge devices for immediate real-time processing and enhanced privacy.

21. The method of claim 20, further including dynamically alocating processing tasks between cloud, fog, and edge computing layers based on computational load, latency requirements, and privacy considerations.
